(19) United States
(12) Patent Application Publication
(10) Pub. No.: US 2020/0355681 A1
(43) Pub. Date: Nov. 12, 2020

(54) RAPID QUANTITATIVE ASSAY TO ASSESS DURATION OF INFECTION

(71) Applicant: SEDIA BIOSCIENCES CORPORATION, Beaverton, OR (US)

(72) Inventors: Ronald W. Mink, Beaverton, OR (US); Matthew Quinn, Beaverton, OR (US); Vijaya K. Mokkapati, Beaverton, OR (US)

(73) Assignee: SEDIA BIOSCIENCES CORPORATION, Beaverton, OR (US)

(21) Appl. No.: 16/965,989

(22) PCT Filed: Jan. 30, 2019
(86) PCT No.: PCT/US19/15822

§ 371 Date: Jul. 29, 2020

Related U.S. Application Data
(60) Provisional application No. 62/625,281, filed on Feb. 1, 2018.

(51) Int. Cl. G01N 33/558; G01N 33/569; G01N 33/68

(52) U.S. Cl. CPC G01N 33/558; G01N 33/56988; G01N 2333/162; G01N 2333/161; G01N 33/6854

(57) ABSTRACT

The present disclosure relates to systems and methods for assessing viral, e.g., HIV, infection duration in a subject. More specifically, the present disclosure relates to for assessing viral, e.g., HIV, infection duration in a subject using, inter alia, a reader configured to measure both the number of signal pixels and the intensities of signal pixels to generate a quantitative signal readout that is used to assess average antibody avidity of an anti-viral antibody, e.g., an anti-HIV antibody, in a sample liquid and/or viral, e.g., HIV, infection duration in a subject.

### CROSS-REFERENCE TO RELATED APPLICATION

The present application claims priority to U.S. provisional patent application No. 62/625,281, filed on Feb. 1, 2018, the disclosure of which is incorporated by reference in its entirety for all purposes.

### FIELD

The present disclosure relates to systems and methods for assessing viral, e.g., HIV, infection duration in a subject. More specifically, the present disclosure relates to systems and methods for assessing viral, e.g., HIV, infection duration in a subject using, inter alia, a reader configured to measure both the number of signal pixels and the intensities of signal pixels to generate a quantitative signal readout that is used to assess average antibody avidity of an anti-viral antibody, e.g., an anti-HIV antibody, in a sample liquid and/or viral, e.g., HIV, infection duration in a subject.

### BACKGROUND

The assay described by Granade et al (1) takes a 2-line lateral flow immunochromatographic assay (LFICA) and adds a third reaction line (which we now refer to as “incidence line” or “recency line”) as a binary indicator to distinguish recent from long term infections, using a reduced (relative to the typically saturated diagnostic line) amount of HIV-1 antigen (specifically a multi-clade recombinant gp41 construct) striped and affixed onto the membrane anterior to the diagnostic and control lines (relative to reagent flow) at limiting concentration relative to the amount of antibody in the sample. This sets up a capture line for antibody, which, because it is limiting and because transport of antibody in the specimen added to the assay crosses this line in a very short, transitory manner, is captured primarily based on proportional retention of higher avidity antibodies (where lower avidity antibodies tend not to be captured but continue to migrate past. The Granade article describes the binary assay as an extension of “the concept of a limiting-antigen-based avidity measurement [ . . . ] extended from the enzyme immunoassay format to a rapid, lateral flow test device . . . ” but did not correlate the assay's results to an avidity assay or to a determined mean duration of recent infection (MDRI). Granade did compare it to another assay that differentiates recent from long-term infections based in change in HIV-1 antibody titers associated with disease progression (HIV-1 BED Enzyme Immunoassay or “BED” Assay (2)) asserting a similar MDRI value for the cutoff as that obtained with the BED Assay. The BED Assay, however, functions by estimating MDRI, based on the proportion of HIV-1 antibodies out of total antibodies in a specimen (with low percent HIV-1 positive in early infections, and higher percent in later infections). The BED Assay and HIV-1 positive antibody proportion has been determined to be less accurate in estimating duration of infection or MDRI than measurement of antibody avidity in general, and the HIV-1 Limiting Antigen Avidity EIA specifically (3, 4). US 2017/0307613 A1 discloses a method and its variations for simultaneous detection of antibodies against two or more antigens of human immunodeficiency virus (HIV) and determination of approximate time (duration) post HIV infection, thereby confirming the infection, and determination of recency of an HIV infection. (18)

There is a need for assays for assessing viral, e.g., HIV, infection duration in a subject. The present disclosure addresses this and other related needs.

### BRIEF SUMMARY

In one aspect, disclosed herein is a system for assessing viral, e.g., HIV, infection duration in a subject, which system comprises: a) a lateral flow test device comprising a porous matrix that comprises, from upstream to downstream, a sample application site configured to receive a sample liquid from a subject, and a first test location comprising an immobilized first binding reagent that specifically binds to an anti-viral antibody, e.g., anti-HIV antibody, in said sample liquid having a first average antibody avidity, wherein, relative to said anti-viral antibody, e.g., anti-HIV antibody, in said sample liquid, said first binding reagent is limiting and said anti-viral antibody is in excess, and said sample liquid flows laterally along said lateral flow test device and passes said first test location to form a first detectable signal comprising multiple signal pixels; and b) a reader configured to measure both the number of said signal pixels and the intensities of said signal pixels in said first detectable signal to generate a first quantitative signal readout that is used to assess average antibody avidity of said anti-viral antibody, e.g., anti-HIV antibody, in said sample liquid and/or viral, e.g., HIV, infection duration in said subject.

In another aspect, disclosed herein is a method for assessing viral, e.g., HIV, infection duration in a subject, which method comprises: a) contacting a sample from a subject with a system described above, wherein said liquid sample is applied to a site of said lateral flow test device upstream of said first test location; b) transporting an anti-viral antibody, e.g., an anti-HIV antibody, if present in said liquid sample, and a labeled reagent to said first test location to form a first detectable signal at said first test location, said first detectable signal comprising multiple signal pixels; and c) measuring both the number of said signal pixels and the intensities of said signal pixels in said first detectable signal using said reader to generate a first quantitative signal; and d) assessing average antibody avidity of said anti-viral antibody, e.g., anti-HIV antibody, in said sample liquid and/or viral, e.g., HIV, infection duration in said subject based on said first quantitative signal.

### BRIEF DESCRIPTION OF THE DRAWINGS

FIG. 1 illustrates an exemplary lateral flow test device that is included in an exemplary system for assessing viral, e.g., HIV, infection duration. The device shown is a test strip comprising several overlapping materials mounted on a Backer, e.g., with adhesive, through which a liquid sample comprised of a sample buffer mixed with a blood or other specimen, migrates through a Sample Pad (conditioning the specimen), a Conjugate Pad (where a colored marker identifying antibody binds to antibody in the sample) to produce a reaction on a Membrane on three reagent lines, i.e. in order of Sample Flow 1) a Recency Line indicated of the duration of HIV infection, 2) a Diagnostic Line indicating presence of HIV antibody and thus infection, and 3) a Control Line, indicative of proper test functionality and a valid sample. The sample continues to migrate to an Absorbent Wick at the far end of the test device, which absorbs and draws through the entire test strip the sample, clear and to facilitate visualization of the three reactive lines on the Membrane. A Cover, e.g., a Gold Pad Cover and Wick Cover may optionally be mounted over the Sample Pad/Conjugate Pad and the Absorbent Pad, respectively, for aesthetic appearance or to facilitate identification of the test or a place to mark identification of test sample.

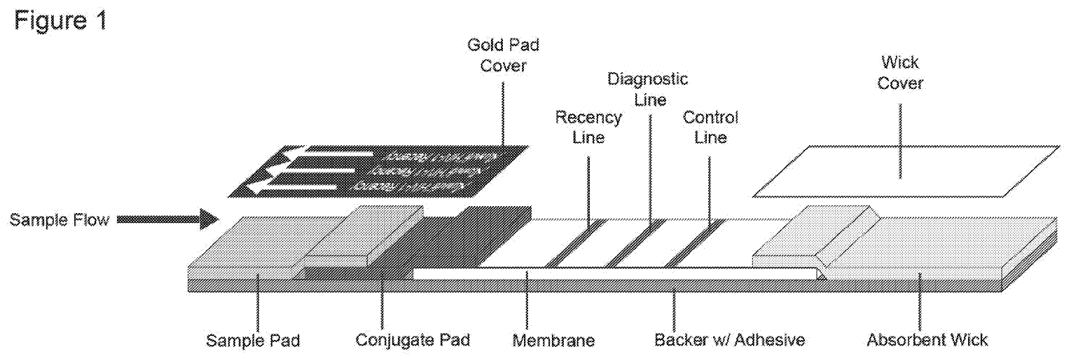

FIG. 2 illustrates an exemplary lateral flow test device strip similar to that in FIG. 1, but alternatively mounted in a plastic housing except that the strip, optionally, does not include a Gold Pad Cover or a Wick Cover as an exemplary system for assessing viral, e.g., HIV, infection duration. Such a device could provide further advantages over the device strip shown in FIG. 1 in that exposure to the user or immediate environment of reagents and sample would be minimized, the plastic housing could be additionally labeled to clearly identify the different reaction strips, additional trade dress could provide additional information to the user, and additional space for marking identification of the sample on the device would be present. The exemplary design also enables a housing that could substitute for a carriage or adapter that may otherwise be required for reading the device strip in an automated reader that measures strip reaction upon insertion of the test device into said reader.

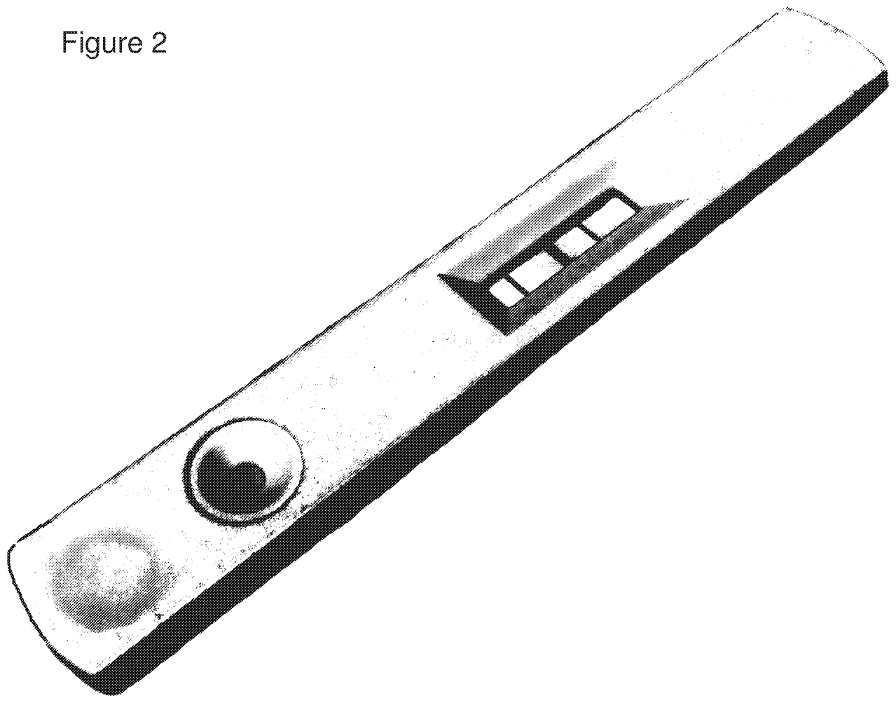

FIG. 3 illustrates an exemplary Asanté™ HIV-1 Rapid Recency™ Assay Response relative to HIV-1 Antibody Avidity and Estimated Mean Duration of Recent Infection. The reaction of the Recency Line on the invention's device strip is measured by a suitable reader instrument measuring integrated pixel density units (IPDU) of the “recency” line and correlated the logarithrmic value of those measurements (y-axis) to the relative antibody avidity measurements of the sample as determined by a HIV-1 Antibody Limiting Antigen Avidity EIA (normalized OD values or “ODn”)(lower x-axis) or correlate to known durations of infection (upper x-axis) as has previously been determined based on antibody avidity (3, 5).

### DETAILED DESCRIPTION

### A. Definitions

Unless defined otherwise, all technical and scientific terms used herein have the same meaning as is commonly understood by one of ordinary skill in the art to which the present disclosure belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

A plurality of hardware and software based devices, as well as a plurality of different structural components may be used to implement the present disclosure. In addition, it should be understood that embodiments of the present disclosure may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the present disclosure may be implemented in software (e.g., stored on non-transitory computer-readable medium) executable by one or more processors. As such, it should be noted that a plurality of hardware and software based devices, as well as a plurality of different structural components may be utilized to implement the present disclosure.

The use of “including,” “comprising,” or “having” and variations thereof herein is meant to encompass the items listed thereafter and equivalents thereof as well as additional items. The use of any and all examples, or exemplary language (e.g., “such as”) is intended to better illuminate the embodiments and does not pose a limitation on the scope of the claims unless otherwise stated.

As used herein and in the appended claims, the singular forms “a,” “an,” and “the” include plural referents unless the context clearly dictates otherwise. Similarly, when the plural form is used it is to be construed to cover the singular form as the context permits. For example, “a” or “an” means “at least one” or “one or more.” Thus, reference to “an analyte” refers to one or more analytes, and reference to “the method” includes reference to equivalent steps and methods disclosed herein and/or known to those skilled in the art, and so forth.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, an “individual” or a “subject” can be any living organism, including humans and other mammals. As used herein, the term “subject” is not limited to a specific species or sample type. For example, the term “subject” may refer to a patient, and frequently a human patient. However, this term is not limited to humans and thus encompasses a variety of mammalian or other species. In one embodiment, the subject can be a mammal or a cell, a tissue, an organ or a part of the mammal. Mammals include any of the mammalian class of species, preferably human (including humans, human subjects, or human patients). Mammals include, but are not limited to, farm animals, sport animals, pets, primates, horses, dogs, cats, mice and rats.

As used herein, the term “sample” refers to anything which may contain an analyte for which an analyte assay is desired. As used herein, a “biological sample” can refer to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as saliva, urine, blood, plasma, serum, semen, stool, sputum, cerebrospinal fluid, synovial fluid, sweat, tears, mucus, amniotic fluid, tissue and organ samples from animals and plants and processed samples derived therefrom. Examples of biological tissues also include organs, tumors, lymph nodes, arteries and individual cell(s).

As used herein, “antibody” refers a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.” An “antibody” may be naturally occurring or man-made such as monoclonal antibodies produced by conventional hybridoma technology, various display methods, e.g., phage display, and/or a functional fragment thereof.

The term “epitope” refers to an antigenic determinant capable of specific binding to an antibody. Epitopes usually or often consist of chemically active surface groupings of molecules such as amino acids or sugar side chains and can have specific three dimensional structural characteristics, as well as specific charge characteristics. Conformational and nonconformational epitopes are distinguished in that the binding to the former but not the latter is lost in the presence of denaturing solvents.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, a “binding reagent” refers to any substance that binds to a target or an analyte with desired affinity and/or specificity. Non-limiting examples of the binding reagent include cells, cellular organelles, viruses, particles, microparticles, molecules, or an aggregate or complex thereof, or an aggregate or complex of molecules. Exemplary binding reagents can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, an aptamer and a complex thereof.

As used herein, the term “specifically binds” refers to the specificity of a binding reagent, e.g., an antibody or an aptamer, such that the binding reagent preferentially binds to a defined target or analyte. A binding reagent “specifically binds” to a target if it binds with greater affinity, avidity, more readily, and/or with greater duration than it binds to other substances. For example, a binding reagent that specifically binds to a target may bind to the target analyte with at least about 10%, at least about 20%, at least about 30%, at least about 40%, at least about 50%, at least about 60%, at least about 70%, at least about 80%, at least about 90% or more, greater affinity as compared to binding to other substances; or with at least about two-fold, at least about five-fold, at least about ten-fold or more of the affinity for binding to a target analyte as compared to its binding to other substances. Recognition by a binding reagent of a target analyte in the presence of other potential interfering substances is also one characteristic of specifically binding. Preferably, a binding reagent, e.g., an antibody or an aptamer, that is specific for or binds specifically to a target analyte, avoids binding to a significant percentage of non-target substances, e.g., non-target substances present in a testing sample. In some embodiments, a binding reagent avoids binding greater than about 90% of non-target substances, although higher percentages are clearly contemplated and preferred. For example, a binding reagent can avoid binding about 91%, about 92%, about 93%, about 94%, about 95%, about 96%, about 97%, about 98%, about 99%, about 99% and about 99.9% or more of non-target substances. In other embodiments, a binding reagent can avoid binding greater than about 10%, 20%, 30%, 40%, 50%, 60%, or 70%, or greater than about 75%, or greater than about 80%, or greater than about 85% of non-target substances.

### B. Systems for Assessing Viral Infection Duration in a Subject

In one aspect, disclosed herein is a system for assessing viral, e.g., HIV, infection duration in a subject, which system comprises: a) a lateral flow test device comprising a porous matrix that comprises, from upstream to downstream, a sample application site configured to receive a sample liquid from a subject, and a first test location comprising an immobilized first binding reagent that specifically binds to an anti-viral antibody, e.g., anti-HIV antibody, in said sample liquid having a first average antibody avidity, wherein, relative to said anti-viral antibody, e.g., anti-HIV antibody, in said sample liquid, said first binding reagent is limiting and said anti-viral antibody is in excess, and said sample liquid flows laterally along said lateral flow test device and passes said first test location to form a first detectable signal comprising multiple signal pixels; and b) a reader configured to measure both the number of said signal pixels and the intensities of said signal pixels in said first detectable signal to generate a first quantitative signal readout that is used to assess average antibody avidity of said anti-viral antibody, e.g., anti-HIV antibody, in said sample liquid and/or viral, e.g., HIV, infection duration in said subject.

In some embodiments, the porous matrix of the lateral flow test device in the present system further comprises, downstream from the first test location, a second test location comprising an immobilized second binding reagent that specifically binds to an anti-viral antibody, e.g., anti-HIV antibody, in said sample liquid having a second average antibody avidity, wherein relative to said anti-viral antibody, e.g., anti-HIV antibody, in said sample liquid, said second binding reagent is in excess and said anti-viral antibody, e.g., anti-HIV antibody, is limiting, said first average antibody avidity is higher than said second average antibody avidity, and the sample liquid flows laterally along the lateral flow test device and passes the first test location to form a first detectable signal, and to passes the second test location to form a second detectable signal; and each of the first detectable signal and the second detectable signal comprise multiple signal pixels.

The lateral flow test device in the present system can comprise the sample application site, the first test location and the second test location in any suitable configuration. In some embodiments, the lateral flow test device comprises a single porous matrix that comprises, from upstream to downstream, the sample application site, the first test location and the second test location. In some embodiments, the lateral flow test device comprises multiple porous matrices that comprise, from upstream to downstream, the sample application site, the first test location and the second test location. In some embodiments, the lateral flow test device comprises two porous matrices, the upstream porous matrix comprises the sample application site and the downstream porous matrix comprises the first test location and the second test location.

The binding reagents can be immobilized at the test locations in any suitable manner. For example, the first binding reagent is covalently immobilized at the first test location. In another example, the first binding reagent is non-covalently immobilized at the first test location. In still another example, the first binding reagent is immobilized at the first test location via a carrier.

The present systems can be configured or used for assessing infection duration of any suitable virus in a subject. In some embodiments, the present systems can be configured or used for assessing infection duration of HIV-1 in a subject. The first binding reagent binds, and preferably specifically binds, to any suitable anti-HIV-1 antibody. For example, the first binding reagent specifically binds to an antibody against HIV-1 Group M, Group N, Group O, Group P. The first binding reagent can specifically bind to an antibody against a HIV-1 envelope or core protein. For example, the first binding reagent can specifically bind to an antibody against a HIV-1 envelope glycoprotein 120 (gp120), envelope glycoprotein 41 (gp41), or viral core protein 24 (p24).

Any suitable first binding reagent, e.g., first binding antigen reagent, that specifically binds to an anti-HIV-1 antibody, preferably that specifically binds to an antigen-antibody binding site of an anti-HIV-1 antibody, can be used. For example, the first binding reagent comprises a polypeptide that specifically binds to an anti-HIV-1 antibody. In another example, the polypeptide that specifically binds to an anti-HIV-1 antibody is a recombinant polypeptide. In still another example, the polypeptide comprises an immunodominant region (IDR) of a HIV-1 envelope or core protein. In yet another example, the polypeptide comprises an immunodominant region (IDR) of a HIV-1 gp120, gp41 or p24.

In some embodiments, The present systems can be configured or used for assessing infection duration of HIV-2 in a subject. The first binding reagent binds, and preferably specifically binds, to any suitable anti-HIV-2 antibody. For example, the first binding reagent specifically binds to an antibody against HIV-2 Group A, B, C, D, E, F, G or H. The binding reagent can specifically bind to an antibody against a HIV-2 envelope or core protein. For example, the first binding reagent specifically binds to an antibody against a HIV-2 envelope glycoprotein 105 (gp105), envelope glycoprotein 125 (gp125), envelope glycoprotein 36 (gp36) or core protein 26 (p26).

Any suitable first binding reagent that specifically binds to an anti-HIV-2 antibody can be used. For example, the first binding reagent comprises a polypeptide that specifically binds to an anti-HIV-2 antibody. In another example, the polypeptide that specifically binds to an anti-HIV-2 antibody is a recombinant polypeptide. In still another example, the polypeptide comprises an immunodominant region (IDR) of a HIV-2 envelope or core protein. In yet another example, the polypeptide comprises an immunodominant region (IDR) of a HIV-2 gp105, gp125, gp36 or p26.

In some embodiments, The present systems can be configured or used for assessing infection duration of HIV-1 in a subject and the porous matrix of the lateral flow test device in the present system further comprises, downstream from the first test location, a second test location comprising an immobilized second binding reagent that specifically binds to an anti-HIV-1 antibody. The second binding reagent can be immobilized at the test locations in any suitable manner. For example, the second binding reagent is covalently immobilized at the second test location. In another example, the second binding reagent is non-covalently immobilized at the second test location. In still another example, the second binding reagent is immobilized at the second test location via a carrier.

The second binding reagent binds, and preferably specifically binds, to any suitable anti-HIV-1 antibody. For example, the second binding reagent specifically binds to an antibody against HIV-1 Group M, Group N, Group O, Group P. The second binding reagent can specifically bind to an antibody against a HIV-1 envelope or core protein. For example, the second binding reagent specifically binds to an antibody against a HIV-1 envelope glycoprotein 120 (gp120), envelope glycoprotein 41 (gp41) or core protein 24 (p24).

Any suitable second binding reagent that specifically binds to an anti-HIV-1 antibody can be used. For example, the second binding reagent comprises a polypeptide that specifically binds to an anti-HIV-1 antibody. In another example, the polypeptide that specifically binds to an anti-HIV-1 antibody is a recombinant polypeptide. In still another example, the polypeptide comprises an immunodominant region (IDR) of a HIV-1 envelope or core protein. In yet another example, the polypeptide comprises an immunodominant region (IDR) of a HIV-1 gp120, gp41 or p24.

In some embodiments, the present systems can be configured or used for assessing infection duration of HIV-2 in a subject and the porous matrix of the lateral flow test device in the present system further comprises, downstream from the first test location, a second test location comprising an immobilized second binding reagent that specifically binds to an anti-HIV-2 antibody. The second binding reagent binds, and preferably specifically binds, to any suitable anti-HIV-2 antibody. For example, the second binding reagent specifically binds to an antibody against HIV-2 Group A, B, C, D, E, F, G or H. The second binding reagent can specifically bind to an antibody against a HIV-2 envelope or core protein. For example, the second binding reagent specifically binds to an antibody against a HIV-2 gp105, gp125, gp36 or p26.

Any suitable second binding reagent that specifically binds to an anti-HIV-2 antibody can be used. For example, the second binding reagent comprises a polypeptide that specifically binds to an anti-HIV-2 antibody. In another example, the polypeptide that specifically binds to an anti-HIV-2 antibody is a recombinant polypeptide. In still another example, the polypeptide comprises an immunodominant region (IDR) of a HIV-2 envelope or core protein. In yet another example, the polypeptide comprises an immunodominant region (IDR) of a HIV-2 gp105, gp125, gp36 or p26.

The first binding reagent and the second binding reagent can specifically bind to an antibody against the same type of HIV or different types of HIV. In some embodiments, the first binding reagent and the second binding reagent specifically bind to an antibody against different types of HIV. For example, the first binding reagent and the second binding reagent specifically bind to an anti-HIV-1 antibody and anti-HIV-2 antibody (or vice versa), respectively.

In some embodiments, both the first binding reagent and the second binding reagent specifically bind to an antibody against the same type of HIV, e.g., an anti-HIV-1 antibody or anti-HIV-2 antibody. For example, both the first binding reagent and the second binding reagent specifically bind to an antibody against HIV-1.

The first binding reagent and the second binding reagent can comprise the same epitope that specifically binds to an antibody against the same type of HIV or different epitopes that specifically bind to an antibody against the same type of HIV. In some embodiments, both the first binding reagent and the second binding reagent comprise the same epitope that specifically binds to an antibody against the same type of HIV, e.g., an anti-HIV-1 antibody or anti-HIV-2 antibody. In some embodiments, the first binding reagent and the second binding reagent comprise different epitopes that specifically bind to an antibody against the same type of HIV, e.g., an anti-HIV-1 antibody or anti-HIV-2 antibody.

The first test location can comprise any suitable amount, level or concentration of an immobilized first binding reagent. In some embodiments, the first test location comprises from about 1 ng/mm to about 100 ng/mm, or any sub-range thereof, of an immobilized first binding reagent, e.g., about 1 ng/mm, 2 ng/mm, 3 ng/mm, 4 ng/mm, 5 ng/mm, 6 ng/mm, 7 ng/mm, 8 ng/mm, 9 ng/mm, 10 ng/mm, 20 ng/mm, 30 ng/mm, 40 ng/mm, 50 ng/mm, 60 ng/mm, 70 ng/mm, 80 ng/mm, 90 ng/mm, or 100 ng/mm of an immobilized first binding reagent.

The second test location can comprise any suitable amount, level or concentration of an immobilized second binding reagent. In some embodiments, the second test location comprises from about 50 ng/mm to about 250 ng/mm, or any sub-range thereof, of an immobilized second binding reagent, e.g., about 50 ng/mm, 60 ng/mm, 70 ng/mm, 80 ng/mm, 90 ng/mm, 100 ng/mm, 110 ng/mm, 120 ng/mm, 130 ng/mm, 140 ng/mm, 150 ng/mm, 160 ng/mm, 170 ng/mm, 180 ng/mm, 190 ng/mm, 200 ng/mm, 210 ng/mm, 220 ng/mm, 230 ng/mm, 240 ng/mm, or 250 ng/mm of an immobilized second binding reagent.

The amount, level or concentration of the immobilized first binding reagent at the first test location can be different from the amount, level or concentration of the second binding reagent at the second test location. In some embodiments, the amount, level or concentration of the immobilized first binding reagent at the first test location is lower than the amount, level or concentration of the second binding reagent at the second test location. In some embodiments, the ratio between the amount, level or concentration of the immobilized second binding reagent at the second test location and the amount, level or concentration of the first binding reagent at the first test location can be from about 2.5:1 to about 50:1, or any sub-range thereof, e.g., about 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 20:1, 30:1, 40:1, or 50:1.

The distance from the bottom of the sample application pad or site to the first test location can be any suitable distance. In some embodiments, the distance from the bottom of the sample application pad to the first test location is from about 37 mm to about 39 mm, or any sub-range thereof, e.g., about 37 mm, 37.5 mm, 38 mm, 38.5 mm, or 39 mm.

The distance from the bottom of the sample application pad or site to the second test location can be any suitable distance. In some embodiments, the distance from the bottom of the sample application pad to the second test location is from about 43 mm to about 45 mm, or any sub-range thereof, e.g., about 43 mm, 43.5 mm, 44 mm, 44.5 mm, or 45 mm.

The ratio between the distance from the bottom of the sample application pad or site to the first test location and the distance from the bottom of the sample application pad or site to the second test location can be any suitable ratio. In some embodiments, the ratio between the distance from the bottom of the sample application pad to the first test location and the distance from the bottom of the sample application pad to the second test location is from about 0.5 to about 1, or any sub-range thereof, e.g., about 0.5, 0.6, 0.7, 0.8, 0.9, or 1.

The first binding reagent can specifically bind to an anti-HIV antibody having any suitable first average antibody avidity. In some embodiments, the first binding reagent specifically binds to an anti-HIV antibody having a first average antibody avidity from about 0.25 normalized OD units (ODn) to about 6.0 ODn as measured by a HIV-1 Limiting Antigen Avidity EIA described in Duong et at., (3), or any sub-range thereof, e.g., about 0.25 ODn, 0.5 ODn, 0.75 ODn, 1 ODn, 1.5 ODn, 2 ODn, 2.5 ODn, 3 ODn, 3.5 ODn, 4 ODn, 4.5 ODn, 5 ODn, 5.5 ODn, or 6 ODn.

The porous matrix can have any suitable form or shape. For example, the porous matrix can be in the form of a strip or a circle. The porous matrix can also have suitable number of elements. For example, the porous matrix can be made of a single element or can comprise multiple elements.

The test device can further comprise a sample application element upstream from and in fluid communication with the matrix. The porous matrix and/or sample application element can be made of any suitable materials, such as nitrocellulose, glass fiber, polypropylene, polyethylene (preferably of very high molecular weight), polyvinylidene fluoride, ethylene vinylacetate, acrylonitrile or polytetrafluoro-ethylene. The porous matrix and the sample application element can comprise the same or different materials.

The test device can further comprise a liquid absorption element downstream from and in fluid communication with the matrix. The liquid absorption element can be made of any suitable materials, such as paper or cellulose materials.

The test device can further comprise a control location comprising means for indicating proper flow of the liquid sample and/or a valid test result. Any suitable means can be used. In one example, the means comprises a binding reagent that binds to a binding reagent with a detectable label that also binds to the analyte, e.g., an anti-viral antibody such as an anti-HIV antibody. In another example, the means comprises a binding reagent that binds to a binding reagent with a detectable label that does not bind to the analyte. In still another example, the means comprises a substance that will generate a detectable signal, e.g., color or electrical signal, once a liquid flow along or through the control location.

In some embodiments, at least a portion of the matrix is supported by a solid backing. In other embodiments, half, more than half or all portion of the matrix is supported by a solid backing. The solid backing can be made of any suitable material, e.g., solid plastics. If the test device comprises electrode or other electrical elements, the solid backing should generally comprise non-conductive materials.

In some embodiments, a labeled reagent can be dried on the test device and the dried labeled reagent can be redissolved or resuspended by a liquid, e.g., a sample liquid and/or additional liquid, and transported laterally through the test device to generate readout, control and/or other signals. For example, a portion of the matrix, upstream from the first test location, the second test location, and/or a control location, can comprise a dried, labeled reagent, the labeled reagent capable of being moved by a liquid sample and/or a further liquid to the first test location, the second test location, and/or a control location, to generate a detectable signal. The dried, labeled reagent can be located at any suitable places on the test device. In one example, the dried, labeled reagent is located downstream from a sample application place on the test device. In another example, the dried, labeled reagent is located upstream from a sample application place on the test device. The type of the labeled reagent can be determined based on the intended assay formats. For example, if the test device is to be used in a sandwich assay, the labeled reagent should be capable of binding, and preferably capable of specifically binding, to the analyte, e.g., an anti-viral antibody such as an anti-HIV antibody, or another substance that binds to the analyte. The same labeled reagent can also be used for certain competitive binding assays. For other types of the competitive binding assays, the labeled reagent should be an analyte, e.g., an anti-viral antibody such as an anti-HIV antibody, or an analyte analog linked to a detectable label. In some embodiments, the control location comprises an immobilized third binding reagent that binds to a labeled reagent or an antibody in a sample liquid.

In some embodiments, the test device can further comprise, upstream from the first test location, the second test location, and/or a control location, a conjugate element that comprises a dried, labeled reagent, the labeled reagent being capable of moved by a liquid sample and/or a further liquid to the first test location, the second test location, and/or a control location, to generate a detectable signal. The conjugate element can be located downstream from a sample application place on the test device. The conjugate element can also be located upstream from a sample application place on the test device. In some embodiments, the labeled reagent binds to an analyte, e.g., an anti-viral antibody such as an anti-HIV antibody, in the liquid sample. In other embodiments, the labeled reagent competes with an analyte, e.g., an anti-viral antibody such as an anti-HIV antibody, in the liquid sample for binding to a binding reagent for the analyte at the first test location and/or the second test location.

Any suitable labeled reagent can be used. In some embodiments, the labeled reagent binds, and preferably specifically binds, to an anti-viral antibody, e.g., an anti-HIV antibody, in the sample.

Any suitable label can be used. The label can be a soluble label, such as a colorimetric, radioactive, enzymatic, luminescent or fluorescent label. The label can also be a particle or particulate label, such as a particulate direct label, or a colored particle label. Exemplary particle or particulate labels include colloidal gold label, latex particle label, nanoparticle label and quantum dot label. Depending on the specific configurations, the labels such as colorimetric, radioactive, enzymatic, luminescent or fluorescent label, can be either a soluble label or a particle or particulate label. In some embodiments, the label is a soluble label, e.g., a fluorescent label. In some embodiments, the label is a particle label, e.g., a gold or latex particle label.

In some embodiments, the labeled reagent is dried in the presence of a material that stabilizes the labeled reagent, facilitates solubilization or resuspension of the labeled reagent in a liquid, and/or facilitates mobility of the labeled reagent. Any suitable material can be used. For example, the material can be a protein, e.g., a meta-soluble protein, a casein or BSA, a peptide, a polysaccharide, a sugar, e.g., sucrose, a polymer, e.g., polyvinylpyrrolidone (PVP-40), a gelatin or a detergent, e.g., Tween-20. See e.g., U.S. Pat. Nos. 5,120,643 and 6,187,598.

The present test devices can be used with any suitable sample liquid. In one example, a sample liquid alone is used to transport the analyte and/or the labeled reagent to the first test location, the second test location, and/or a control location. In another example, a developing liquid is used to transport the analyte and/or the labeled reagent to the first test location, the second test location, and/or a control location. In still another example, both sample liquid and a developing liquid is used to transport the analyte and/or the labeled reagent to the first test location, the second test location, and/or a control location.

In some embodiments, the test device can further comprise a housing that covers at least a portion of the test device, wherein the housing comprises a sample application port to allow sample application upstream from or to the first test location, the second test location, and/or a control location and an optic opening around the first test location, the second test location, and/or a control location to allow signal detection at the first test location, the second test location, and/or a control location. The optic opening can be achieved in any suitable way. For example, the optic opening can simply be an open space. Alternatively, the optic opening can be a transparent cover.

In other embodiments, the housing can cover the entire test device. In still other embodiments, at least a portion of the sample receiving portion of the matrix or the sample application element is not covered by the housing and a sample is applied to the portion of the sample receiving portion of the matrix or the sample application element outside the housing and then transported to the first test location, the second test location, and/or a control location. The housing can comprise any suitable material. For example, the housing can comprise a plastic material, a biodegradable material or a cellulosic material. In another example, the housing, whether in part or in its entirety, can comprise an opaque, translucent and/or transparent material.

The reader in the present systems can comprise an image sensor. Any suitable image sensor can be used. In some embodiments, the image sensor is an active pixel sensor, e.g., a complementary metal-oxide-semiconductor (CMOS) active pixel sensor. The reader in the present systems can comprise any suitable number of image sensor(s) or pixel sensor(s). For example, the reader can comprise a single image sensor or pixel sensor. In another example, the reader can comprise an array of pixel sensors.

The reader in the present systems can have or use any suitable optical format. In some embodiments, the reader has an optical format from about 1/13 inch to about 4/3 inch or any sub-range thereof, e.g., about ⅙ inch, ⅕ inch, ¼ inch, 1/3.6 inch, 1/3.2 inch, ⅓ inch, 1/2.7 inch, 1/2.5 inch, 1/2.3 inch, ½ inch, or ⅔ inch.

The reader in the present systems can have or use any suitable pixel size. In some embodiments, the reader has a pixel size from about 1.1 micrometers to about 8 micrometers or any subrange thereof, e.g. about 1.2 micrometers 1.25 micrometers, 1.4 micrometers, 1.67 micrometers, 1.75 micrometers, 1.9 micrometers, 2.2 micrometers, 2.4 micrometers, 2.8 micrometers, 3.0 micrometers, 3.5 micrometers, 3.75 micrometers, 4.5 micrometers, 4.7 micrometers, 4.8 micrometers, 5.2 micrometers, 5.6 micrometers, or 6.0 micrometers.

The reader in the present systems can have or use any suitable array size. In some embodiments, the reader has an array size from about 1 megapixel to about 5 megapixel, or any sub-range thereof, e.g., about 1 megapixel, 2 megapixel, 3 megapixel, 4 megapixel, or 5 megapixel.

The reader in the present systems can have or use any suitable reading time. In some embodiments, the reader has a reading time from about 1 second to about 30 seconds, or any sub-range thereof, e.g., about 1 second, 2 seconds, 3 seconds, 4 seconds, 5 seconds, 6 seconds, 7 seconds, 8 seconds, 9 seconds, 10 seconds, 12 seconds, 15 seconds 17 seconds, 20 seconds, 25 seconds or 30 seconds.

The quantitative signal readout can have or use any suitable unit. In some embodiments, the first quantitative signal readout uses integrated pixel density unit (IPDU). The reader in the present systems can be configured to generate the first quantitative signal readout with any suitable range of linearity. In some embodiments, the reader is configured to generate the first quantitative signal readout with a range of linearity from about 1 IPDU to about 10,000,000 IPDU, or any sub-range thereof, e.g., about 1 IPDU, 5 IPDU, 10 IPDU, 50 IPDU, 100 IPDU, 500 IPDU, 1,000 IPDU, 5,000 IPDU, 10,000 IPDU, 50,000 IPDU, 100,000 IPDU, 500,000 IPDU, 1,000,000 IPDU, 2,000,000 IPDU, 3,000,000 IPDU, 4,000,000 IPDU, 5,000,000 IPDU, 6,000,000 IPDU, 7,000,000 IPDU, 8,000,000 IPDU, 9,000,000 IPDU, or 10,000,000 IPDU.

In some embodiments, the second quantitative signal readout uses integrated pixel density unit (IPDU). The reader in the present systems can be configured to generate the second t quantitative signal readout with any suitable range of linearity. In some embodiments, the reader is configured to generate the second quantitative signal readout with a range of linearity from about 1 IPDU to about 10,000,000 IPDU, or any sub-range thereof, e.g., about 1 IPDU, 5 IPDU, 10 IPDU, 50 IPDU, 100 IPDU, 500 IPDU, 1,000 IPDU, 5,000 IPDU, 10,000 IPDU, 50,000 IPDU, 100,000 IPDU, 500,000 IPDU, 1,000,000 IPDU, 2,000,000 IPDU, 3,000,000 IPDU, 4,000,000 IPDU, 5,000,000 IPDU, 6,000,000 IPDU, 7,000,000 IPDU, 8,000,000 IPDU, 9,000,000 IPDU, or 10,000,000 IPDU.

The present system or test device can further comprise a liquid container. The liquid container can comprise any suitable liquid and/or reagent. For example, the liquid container can comprise a developing liquid, a wash liquid and/or a labeled reagent.

The present system or test device can further comprise machine-readable information, e.g., a barcode. The barcode can comprise any suitable information. In some embodiments, the barcode comprises lot specific information of the present system or test device, e.g., lot number of the present system or test device. In other embodiments, the machine-readable information is comprised in a storage medium, e.g., a (radio-frequency identification) RFID device. The RFID device can comprise any suitable information. For example, the RFID device comprises lot specific information, information on a liquid control or information to be used for quality control purpose.

The present system can be configured or used to assess any suitable infection duration. In some embodiments, the present system can be configured or used to assess viral infection duration, e.g., HIV infection duration, from about 10 days to about 450 days, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days.

### C. Systems for Assessing Viral Infection Duration in a Subject

In another aspect, disclosed herein is a method for assessing viral, e.g., HIV, infection duration in a subject, which method comprises: a) contacting a sample from a subject with a system described in above Section B., wherein said liquid sample is applied to a site of said lateral flow test device upstream of said first test location; b) transporting an anti-viral antibody, e.g., an anti-HIV antibody, if present in said liquid sample, and a labeled reagent to said first test location to form a first detectable signal at said first test location, said first detectable signal comprising multiple signal pixels; and c) measuring both the number of said signal pixels and the intensities of said signal pixels in said first detectable signal using said reader to generate a first quantitative signal; and d) assessing average antibody avidity of said anti-viral antibody, e.g., anti-HIV antibody, in said sample liquid and/or viral, e.g., HIV, infection duration in said subject based on said first quantitative signal.

In some embodiments, the liquid sample and the labeled reagent are premixed to form a mixture and the mixture is applied to the test device. For example, the labeled reagent can be provided or stored in a liquid and then can be premixed with a sample liquid to form a mixture and the mixture is applied to the test device. In another example, the labeled reagent can be dried in a location or container not in fluid communication with the test device, e.g., in a test tube or well such as a microtiter plate well. In use, the sample liquid can be added to the container, e.g., the test tube or well, to form the mixture and the mixture can then be applied to the test device.

In some embodiments, the present method can further comprise a washing step after the mixture is applied to the lateral flow test device. The washing step can be conducted in any suitable manner. For example, the washing step comprises adding a washing liquid after the mixture is applied to the lateral flow test device. In another example, the lateral flow test device comprises a liquid container comprising a washing liquid and the washing step comprises releasing the washing liquid from the liquid container.

In other embodiments, the test device comprises a dried labeled reagent before use and the dried labeled reagent is solubilized or resuspended, and transported to the first test location, the second test location, and/or a control location by the liquid sample and/or other liquid. The dried labeled reagent can be located at any suitable location on the test device. For example, the dried labeled reagent can be located downstream from the sample application site, and the dried labeled reagent can be solubilized or resuspended, and transported to the first test location, the second test location, and/or a control location by the liquid sample and/or other liquid. In another example, the dried labeled reagent can be located upstream from the sample application site, and the dried labeled reagent can be solubilized or resuspended, and transported to the first test location, the second test location, and/or a control location by another liquid.

In some embodiments, the labeled reagent is solubilized or resuspended, and transported to the first test location, the second test location, and/or a control location by the liquid sample alone. In other embodiments, the analyte and/or labeled reagent is solubilized or resuspended, and transported to the first test location, the second test location, and/or a control location by another liquid. In still other embodiments, the analyte and/or labeled reagent is solubilized or resuspended, and transported to the first test location, the second test location, and/or a control location by both the sample liquid and another liquid, e.g., a developing liquid.

The present method can be used for assessing viral, e.g., HIV, infection duration in any suitable subject. In some embodiments, the present method can be used for assessing viral, e.g., HIV, infection duration in a mammal, e.g., a human or a non-human mammal. In other embodiments, the present method can be used for assessing viral, e.g., HIV, infection duration in a bird, e.g., a chicken. In still other embodiments, the present method can be used for assessing viral, e.g., HIV, infection duration in a reptile or a fish.

The present methods can be used for assessing viral, e.g., HIV, infection duration in a subject using any suitable sample. In some embodiments, the liquid sample can be body fluid sample, such as a whole blood, a serum, a plasma, a urine sample or an oral fluid. Such body fluid sample can be used directly or can be processed, e.g., enriched, purified, or diluted, before use. In other embodiments, the liquid sample can be a liquid extract, suspension or solution derived from a solid or semi-solid biological material such as a phage, a virus, a bacterial cell, an eukaryotic cell, a fugal cell, a mammalian cell, a cultured cell, a cellular or subcellular structure, cell aggregates, tissue or organs. In some embodiments, the sample liquid is obtained or derived from a mammalian or human source. In other embodiments, the sample liquid is a clinical sample, e.g., a human or animal clinical sample. In still other embodiments, the sample liquid is a man-made sample, e.g., a standard sample for quality control or calibration purposes.

The present methods can be used for assessing infection duration of any suitable virus in a subject. In some embodiments, the present methods can be used for assessing infection duration of HIV-1 in a subject. The first binding reagent binds, and preferably specifically binds, to any suitable anti-HIV-1 antibody. For example, the first binding reagent specifically binds to an antibody against HIV-1 Group M, Group N, Group O, Group P. The first binding reagent can specifically bind to an antibody against a HIV-1 envelope or core protein. For example, the first binding reagent can specifically bind to an antibody against a HIV-1 envelope glycoprotein 120 (gp120), envelope glycoprotein 41 (gp41), or viral core protein 24 (p24).

In some embodiments, the present methods can be used for assessing infection duration of HIV-2 in a subject. The first binding reagent binds, and preferably specifically binds, to any suitable anti-HIV-2 antibody. For example, the first binding reagent specifically binds to an antibody against HIV-2 Group A, B, C, D, E, F, G or H. The binding reagent can specifically bind to an antibody against a HIV-2 envelope or core protein. For example, the first binding reagent specifically binds to an antibody against a HIV-2 envelope glycoprotein 105 (gp105), envelope glycoprotein 125 (gp125), envelope glycoprotein 36 (gp36) or core protein 26 (p26).

The first binding reagent can specifically bind to an anti-HIV antibody having any suitable first average antibody avidity. In some embodiments, the first binding reagent specifically binds to an anti-HIV antibody having a first average antibody avidity from about 0.25 normalized OD units (ODn) to about 6.0 ODn as measured by a HIV-1 Limiting Antigen Avidity EIA described in Duong et at., (3), or any sub-range thereof, e.g., about 0.25 ODn, 0.5 ODn, 0.75 ODn, 1 ODn, 1.5 ODn, 2 ODn, 2.5 ODn, 3 ODn, 3.5 ODn, 4 ODn, 4.5 ODn, 5 ODn, 5.5 ODn, or 6 ODn. The second binding reagent can specifically bind to an anti-HIV antibody having any suitable second average antibody avidity. In some embodiments, the second binding reagent specifically binds to an anti-HIV antibody having a second average antibody avidity from about 0.25 normalized OD units (ODn) to about 6 ODn as measured by a HIV-1 Limiting Antigen Avidity EIA described in Duong et at. (3), e.g., about 0.25 ODn, 0.50 ODn, 0.75 ODn, 1.00 ODn, 1.25 ODn, 1.50 ODn, 1.75 ODn, 2.00 ODn, 2.25 ODn, 2.50 ODn, 2.75 ODn, 3.00 ODn, 3.25 ODn, 3.50 ODn, 3.75 ODn, 4.00 ODn, 4.25 ODn, 4.50 ODn, 4.75 ODn, 5.00 ODn, 5.25 ODn, 5.50 ODn, 5.75 ODn, or 6.00 ODn, or any subrange thereof.

The quantitative signal readout in the present methods can have or use any suitable unit. In some embodiments, the first quantitative signal readout uses integrated pixel density unit (IPDU). The reader in the present systems can be used to generate the first quantitative signal readout with any suitable range of linearity. In some embodiments, the reader is used to generate the first quantitative signal readout with a range of linearity from about 1 IPDU to about 10,000,000 IPDU, or any sub-range thereof, e.g., about 1 IPDU, 5 IPDU, 10 IPDU, 50 IPDU, 100 IPDU, 500 IPDU, 1,000 IPDU, 5,000 IPDU, 10,000 IPDU, 50,000 IPDU, 100,000 IPDU, 500,000 IPDU, 1,000,000 IPDU, 2,000,000 IPDU, 3,000,000 IPDU, 4,000,000 IPDU, 5,000,000 IPDU, 6,000,000 IPDU, 7,000,000 IPDU, 8,000,000 IPDU, 9,000,000 IPDU, or 10,000,000 IPDU.

In some embodiments, the second quantitative signal readout uses integrated pixel density unit (IPDU). The reader in the present systems can be used to generate the second quantitative signal readout with any suitable range of linearity. In some embodiments, the reader is used to generate the second quantitative signal readout with a range of linearity from about 1 IPDU to about 10,000,000 IPDU, or any sub-range thereof, e.g., about 1 IPDU, 5 IPDU, 10 IPDU, 50 IPDU, 100 IPDU, 500 IPDU, 1,000 IPDU, 5,000 IPDU, 10,000 IPDU, 50,000 IPDU, 100,000 IPDU, 500,000 IPDU, 1,000,000 IPDU, 2,000,000 IPDU, 3,000,000 IPDU, 4,000,000 IPDU, 5,000,000 IPDU, 6,000,000 IPDU, 7,000,000 IPDU, 8,000,000 IPDU, 9,000,000 IPDU, or 10,000,000 IPDU.

The present methods can be used for assessing viral, e.g., HIV, infection duration in a subject using any suitable manner. In some embodiments, the viral infection duration, e.g., HIV infection duration, in the subject can be assessed by comparing the first quantitative signal to a predetermined correlation between the viral infection duration, e.g., HIV infection duration, and a reference quantitative signal. In other embodiments, the viral infection duration, e.g., the HIV infection duration, in the subject can be assessed by comparing the first quantitative signal to a reference average antibody avidity that has a predetermined correlation between the viral infection duration, e.g., HIV infection duration, and the reference average antibody avidity.

The present methods can be used to assess any suitable infection duration. In some embodiments, the present methods can be used to assess viral infection duration, e.g., HIV infection duration, from about 10 days to about 450 days of mean duration of recent infection (MDRI) measured as time since seroconversion, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days of mean duration of recent infection (MDRI) measured as time since seroconversion.

In some embodiments, the present methods can be used to assess incidence of HIV infections in a population based on a predetermined MDRI cutoff value, e.g., to distinguish recent from long-term infections e.g., HIV infection duration, from about 10 days to about 450 days of mean duration of recent infection (MDRI) measured as time since seroconversion, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days of mean duration of recent infection (MDRI) measured as time since seroconversion. For example, the present methods can be used to assess incidence of HIV infections in a population based on multiple predetermined MDRI cutoff values by identifying recent infections below a specified cutoff, e.g., those cutoffs ranging from about 10 days to about 450 days of mean duration of recent infection (MDRI) measured as time since seroconversion, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days of mean duration of recent infection (MDRI) measured as time since seroconversion.

The present methods can be used to assess any suitable infection duration. In some embodiments, the present methods can be used to assess viral infection duration, e.g., HIV infection duration, from about 10 days to about 450 days, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days.

The present methods can be used for any suitable purposes. In some embodiments, the present methods can be used to assess viral infection duration, e.g., HIV infection duration, in a subject with a false recency rate (FRR) of less than 10% relative to a given MDRI. In some embodiments, the present methods can be used to assess viral infection duration, e.g., HIV infection duration, in a subject with a false recency rate (FRR) of less than 9%, 8%, 7%, 6%, 5%, 4%, 3%, 2%, 1%, 0.5%, or 0.1% relative to a given MDRI.

The present methods can comprise any suitable additional steps. In some embodiments, the present methods can further comprise treating a subject that has been infected with a virus. For example, the present methods can further comprise treating a subject that has been infected that has been infected with HIV in the past about 10 days to about 450 days, or any sub-range thereof, e.g., about 10 days, 20 days, 30 days, 40 days, 50 days, 60 days, 70 days, 80 days, 90 days, 100 days, 150 days, 200 days, 250 days, 300 days, 350 days, 400 days, or 450 days.

### D. Exemplary Embodiments

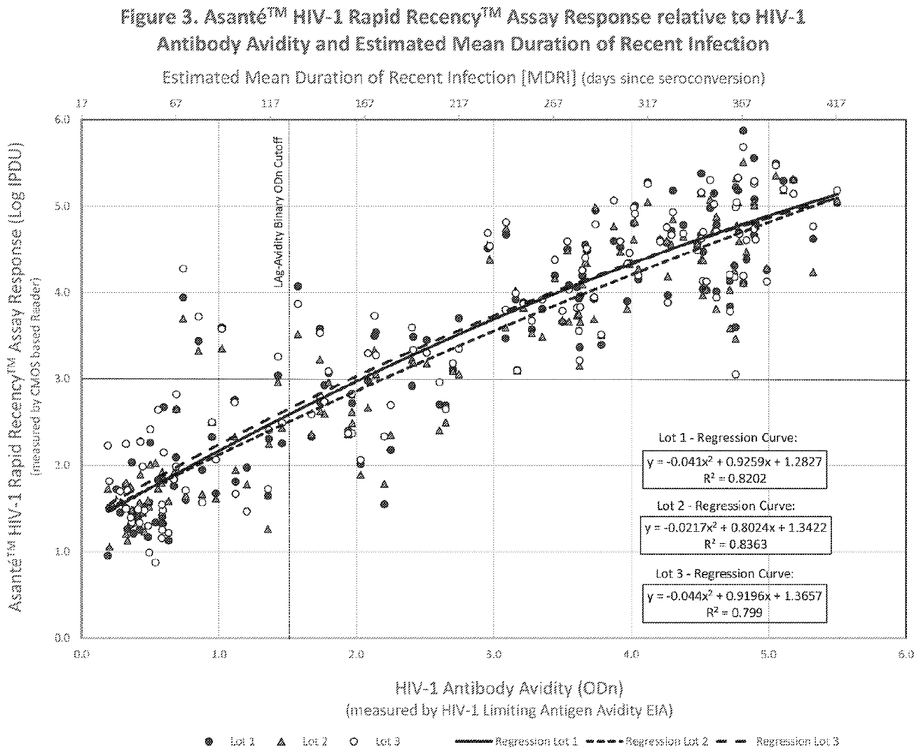

In some embodiments, the present disclosure is directed to an enhancement or improvement over a prior existing rapid colloidal gold lateral flow immunochromatographic assay (LFICA) described by Granade et al (1) to differentiate recent versus long-term infections, using a modification of a diagnostic LFICA characterized by the addition of a third reaction line (now referred to as the “incidence line” or more accurately “recency line” because it is used to estimate recency of infection) which is in addition to the 2 line diagnostic test (with one control line and one diagnostic line). The original assay is a binary assay identifying HIV infections as either “recent” or “long-term”, not quantitating a variable duration of infection based on the visual absence or presence of the recency line. The present assay enhances the assay by allowing the user to actually estimate the duration of infection of any specimen probabilistically, expanding the utility of the assay beyond a simple “recent/long-term” result by incorporating an independent instrument reading of the size and intensity of the recency line and correlating with antibody avidity as measured by a HIV-1 Limiting Antigen Avidity EIA, an assay designed to measure HIV-1 antibody avidity, and whose relationship between HIV-1 antibody avidity of a blood/serum or plasma sample from a patient and the duration of infection in the patient, or the “mean duration of recent infection” (or “MDRI”) has been established. (5). This requires screening and quantitation of the recency line, logarithmic transformation of the quantitative measurements of pixelated color (both number of pixels and intensity on a digital CCR camera based system) to logarithmic values, correlated with antibody avidity values in the LAg-Avidity EIA associated with known MDRI values. (See FIG. 3.)

Enhancement/Improvement

In some embodiments, studies performed by Sedia Biosciences in commercializing the binary assay as conceived by Granade et al, (1) and developing the present assay, however, have correlated the results of the third, recency line to antibody avidity as measured by the HIV-1 LAg-Avidity EIA, and by extension (since the LAg-Avidity EIA normalized ODn values have been correlated to various MDRI values) to duration of HIV-1 infection. One can still maintain a binary assay to measure against a cutoff corresponding to a fixed MDRI, where higher avidity antibodies captured tend, if present in sufficient quantities, to form a visible line, while lower avidity antibodies tend not to be captured, decreasing the likelihood of a visible line. Further by adjusting the amount of antigen striped on the solid phase, the cutoff or threshold of avidity of the antibodies captured can be adjusted such that either a typically higher or lower average avidity of antibodies are captured and form a visible line corresponding to a higher or lower fixed MDRI. In the present studies, we established a relationship between antibody avidity and MDRI in the case of HIV-1 infections. We believe this is also applicable to other diseases in which a humoral (antibody) response is elicited.

The Granade et al version of the assay (1) is used to establish a cutoff to distinguish between recent vs. long term infections (with a cutoff or MDRI estimation of perhaps 4-6 months). The Granade et al version of the assay (1) is not a quantitative measurement using the recency line to establish the ability to measure a variable duration of infection by quantitative measurement of the recency line. The Granade et al version of the assay (1) does not use a reader, as many commercial readers weren't either sensitive enough (as we have found) or didn't have an adequate range of linearity over which to gain an accurate quantitation of the recency line reaction that could be correlated to anything. In some embodiments, we showed that there could be a quantitative correlation of instrument interpretation of that line to the avidity response of the antibody (FIG. 3). When the quantitative measurement of the recency line on an exemplary reader (the Detekt RDS-1500 Pro) are transformed to corresponding logarithmic values, and those values compared to the Avidity values obtained with the same samples using the LAg-Avidity EIA, a 2^{nd} order polynomial regression with high regression coefficient (R>0.8) is obtained. Those avidity values, as measured by LAg-Avidity EIA, also correspond to known MDRIs enabling the translation of recency line results in the assay, quantitatively to a corresponding duration of infection.

Instrument Reader

In some embodiments, the reader used in our studies is the Detekt RDS-1500 (Detekt Biomedical LLC, Austin Tex., www.idetekt.com). The reader uses a Linear CCD sensor with a 630 nm LED Lamp light source and a 33 MHz Motorola DragonBall-VZ microprocessor with 16 MB SDRAM and 4 MB Flash memory. The reader contains an FSTN (TDF) 4 bit grayscale LCD display (160×160 display). Additional information is included on the operation of the reader, which is based on the Model IDV-BCS1 ID::VERIFI Barcode Scanner (Aceeca, Christchurch, New Zealand. www.aceeca.com). The software for the reader is designed to identify the location of the striped reaction lines on test strips inserted into the reader where the image sensor is located and reads only those areas providing a pixelated intensity measurement (Resolution min. 0.127 mm). The output as provided by the manufacturer must be transformed to their log₁₀ values to create a 2nd order polynomial correlation to the LAg-Avidity EIA values (FIG. 3).

Exemplary Utilities

Assays for distinguishing recent vs. long-term infections were initially developed for epidemiologists who sought to develop a way to identify “new infections” in a population in order for them to estimate the incidence of HIV infections. Incidence is the rate of new cases of a disease within a certain period of time. Since HIV is never cured and simple diagnostic tests do not enable one to tell which patients are new cases vs. long term cases, the most accurate way to establish incidence is to monitor a longitudinal cohort of negative subjects at risk for infection over a period of time and determine how many become positive. This can be extremely costly and time consuming, particularly in low prevalence settings, and has its own built in biases in terms of population selection, subject participation and follow through, and other considerations. Developing “recency” assays to identify recent vs. long term infections allows one to determine in the laboratory, by testing a pool of subjects, an estimation of incidence rates. The rapid test version of these laboratory assays has the further advantage of being simpler to use (and not requiring skilled technicians to perform), not required complex, expensive lab infrastructure, can be taken to the point of contact, can be stored at ambient condition (no cold chain storage required), etc. However LFICA tests typically are not measured quantitatively, so in the case of recency assays, the user has only one choice of MDRI to use to estimate incidence rates, determined by the manufacturer of the assay. This can be problematic when an assay uses too short of an MDRI such that a very large number of specimens must be tested to get a statistically valid number of “recent” infections to accurately estimate the incidence rate, or when the MDRI is so long as to increase the likelihood of false recent infections, particularly in a higher prevalence population. With a quantitative recency assay described in the present disclosure, one can select a cutoff for a given study that best suits the investigator's purpose.

A quantitative recency assay described in the present disclosure has additional applications, which may have need for different cutoffs, and thus different MDRI's than a single fixed cutoff predetermined by the assay manufacturer. In recent years, there has been a growing call to more aggressively identify individuals recently infected and target them for early and assertive treatment as growing evidence suggests that zealous intervention in early stage HIV infections is critical to controlling the epidemic (6-8). Recently infected persons are typically the most infectious individuals with relatively high viral loads (9) and as a result have been found to be the source of typically 40-50% and, in some reports, up to 90% of all HIV-1 transmissions (10-12). Indeed, the ability to identify and triage recently infected individuals with HIV has been characterized as a “clinical and public health emergency” (12). Aggressive intervention of new infections not only reduces the high risk of transmission at this stage, but increases the chances to prevent establishment of viral reservoirs where antiviral agents may have diminished effectiveness. Blocking the establishment of such viral reservoirs may ultimately be important in the eventual development of a “cure”, such as a sustained viral remission after discontinuation of antiviral therapy (13). The cutoff suitable for identifying highly infectious early infections, or identifying clinical patients for targeted therapeutic treatment may not be the same for all situations or the same as is optimal for HIV-1 incidence estimation. Having an assay that can measure a variable MDRI lends greater flexibility to this type of assay, and broader utility. Several publications reporting the possible use of such functional cures have cited the need for such treatments, if they are to be effective, be preferentially used in persons that have been recently infected (13-17) although there is no easy method to measure how recently a person has been infected without elaborate laboratory testing. Availability of a rapid simple test suitable for use at the point of first contact of individuals infected with HIV, such as the one described in the present disclosure, can be used to identify suitable candidates in conjunction with the development of and treatment by new functional cures in development for HIV.

Some embodiments of the present disclosure focus on identifying recent vs. long term infections of HIV-1. However, quantitative recency assay described in the present disclosure can also be used for many other types of infections, particularly for those that may not be quickly or readily “curable” and for which the discrimination of early vs late infections is important, such as Hepatitis C, Hepatitis B, dengue fever, etc.

### E. Example

### Example 1. Asanté™ HIV-1 Rapid Recency™ Assay Response Relative to HIV-1 Antibody Avidity and Estimated Mean Duration of Recent Infection

FIG. 3 illustrates an exemplary Asanté™ HIV-1 Rapid Recency™ Assay Response relative to HIV-1 Antibody Avidity and Estimated Mean Duration of Recent Infection. The reaction of the Recency Line on the invention's device strip is measured by a suitable reader instrument measuring integrated pixel density units (IPDU) of the “recency” line and correlated the logarithrmic value of those measurements (y-axis) to the relative antibody avidity measurements of the sample as determined by a HIV-1 Antibody Limiting Antigen Avidity EIA (normalized OD values or “ODn”)(lower x-axis) or correlate to known durations of infection (upper x-axis) as has previously been determined based on antibody avidity (3, 5).

### F. Reference List

Certain cited references are listed below.
1. Granade T C, Nguyen S, Kuehl D S, Parekh B S. 2013. Development of a novel rapid HIV test for simultaneous detection of recent or long-term HIV type 1 infection using a single testing device. AIDS Res Hum Retroviruses 29(1):61-67.
2. Parekh B S, Kennedy M S, Dobbs T, et al. (2002). Quantitative detection of increasing HIV type antibodies after seroconversion: a simple assay for detecting recent HIV infection and estimating incidence. AIDS Res Hum Retroviruses 18:295-307.
3. Duong Y T, Qiu M, De A K, Jackson K, Dobbs T, Kim A A, Nkengasong J N, Parekh B S. (2012). Detection of recent HIV-1 infection using a new limiting-antigen avidity assay: Potential for HIV-1 incidence estimates and avidity maturation studies. PLoS ONE 7(3): e33328. doi10.1371/journal.pone.0033328.
4. Kassanjee R, Pilcher C D, Keating S M, Facente N, McKinney E, Price M A, Martin J N, Little S, Hecht F M, Kailas E G, Welte A, Busch M P, Murphy G. (2014). Independent assessment of candidate HIV incidence assays on specimens in the CEPHIA repository. AIDS 28:239-2449.
5. Duong Y T, Kassanjee R, Welte A, et al. (2015). Recalibration of the Limiting Antigen Avidity EIA to determine mean duration of recent infection in divergent HIV-1 subtypes. PLoS One 10(2):e0114947. doi:10.1371/journal/pone/0114947.
6. Cohen M S, Pilcher C D. (2005). Amplified HIV transmission and new approaches to HIV prevention. J Infect Dis 191:1391-3.
7. Kitahata M M, Gange S J, Abraham A G, Merriman B, Saag M S, Justice A C, Hogg R S, Deeks S G, Eron J J, Brooks J T, Rourke S B, Gill M J, Bosch R J, Martin J N, Klein M B, Jacobson L P, Rodriguez B, Sterling T R, Kirk G D, Napravnik S, Rachlis A R, Calzavara L M, Horberg M A, Silverberg M J, Gebo K A, Goedert J J, Benson C A, Collier A C, Van Rompaey S E, Crane H M, McKaig R G, Lau B, Freeman A M, Moore R D. (2009). Effect of early versus deferred antiretroviral therapy for HIV on survival. New Engl J Med 360(18):1815-261.
8. Cohen M S, Chen Y Q, McCauley M, Gamble T, Hosseinipour M C, Kumarasamy N, Hakim J G, Kumwenda J, Grinsztejn B, Pilotto J H S, Godbole S V, Mehendale S, Chariyalertsak S, Santos B R, Mayer K H, Hoffman I F, Eshleman S H, Piwowar-Manning E, Wang L, Makhema J, Mills L A, de Bruyn G, Sanne I, Eron J, Gallant J, Havlir D, Swindells S, Ribaudo H, Elharrar V, Burns D, Taha T E, Nielsen-Saines K, Celentano D, Essex M, Fleming T R. (2011). Prevention of HIV-1 infection with early antiretroviral therapy. New Engl J Med 365(6):493-505.
9. Pilcher C D, Tien H C, Eron J J, Vernazza P L, Leu S-Y, Steward P W, Goh L-E, Cohen M S. (2004). Brief but efficient: Acute HIV infection and the sexual transmission of HIV. J Infect Dis 189:1785-92.
10. Wawer M J, Gray R H, Sewankambo N K, Serwadda D, Li X, Laeyendecker O, Kiwanuka N, Kigozi G, Kiddugavu M, Lutalo T, Nalugoda F, Wabwire-Mangen F, Meehan M P, Quinn T C. (2005). Rates of HIV-1 transmission per coital act, by stage of HIV-1 infection, in Rakai, Uganda. J Infect Dis 191:1403-9.
11. Brenner B G, Roger M, Routy J-P, Moisi D, Ntemgwa M, Matte C, Baril J-G, Thomas R, Rouleau D, Bruneau J, Leblanc R, Legault M, Tremblay C, Charest H, Wainberg M A, And the Quebec Primary HIV Infection Study Group. (2007). High rates of forward transmission events after acute/early HIV-1 infection. J Infect Dis 195:951-9.
12. Smith M K, Rutstein S E, Powers K A, Fidler S, Miller W C, Eron J J, Cohen M S. (2013) The detection and management of early HIV infection: A clinical and public health emergency. J Acquir. Immune. Defic. Syndr. 63:S187-S199.
13. Ananworanich J, Fauci A S. (2015). HIV cure research: a formidable challenge. J. Virus Eradication 1:1-3.
14. Rosenberg E S, Altfeld M, Poon S H. (2000) Immune control of HIV-1 after early treatment of acute infection. Nature 407:523-526.
15. Sáez-Cirión A, Bacchus C, Hocquelous L, et al. (2013). Post-treatment controllers with a long-term virological remission after the interruption of early initiated antiretroviral therapy ANRS VISCONTI study. PLoS Pathog 9(3):e1003211. doi:10.1371/journal.ppat.1003211.
16. Ananworanich J, McSteen B, Robb M L. (2015). Broadly neutralizing antibody and the HIV reservoir in acute HIV infection: a strategy toward HIV remission? Curr Opin HIV AIDS. 10(3):198-206. doi:10.1097/COH.0000000000000144.
17. Martin A R, Siliciano R F. (2016). Progress toward HIV eradication: Case reports, current efforts and the challenges associated with cure. Ann. Rev. Med. 67:213-228. doi: 10.1146/annurev-med-011514-023043.
18. US 2017/0307613 A1

1. A system for assessing viral, e.g., HIV, infection duration in a subject, which system comprises:
a) a lateral flow test device comprising a porous matrix that comprises, from upstream to downstream,
a sample application site configured to receive a sample liquid from a subject, and
a first test location comprising an immobilized first binding reagent that specifically binds to an anti-viral antibody, e.g., anti-HIV antibody, in said sample liquid having a first average antibody avidity,
wherein, relative to said anti-viral antibody, e.g., anti-HIV antibody, in said sample liquid, said first binding reagent is limiting and said anti-viral antibody is in excess, and said sample liquid flows laterally along said lateral flow test device and passes said first test location to form a first detectable signal comprising multiple signal pixels; and
b) a reader configured to measure both the number of said signal pixels and the intensities of said signal pixels in said first detectable signal to generate a first quantitative signal readout that is used to assess average antibody avidity of said anti-viral antibody, e.g., anti-HIV antibody, in said sample liquid and/or viral, e.g., HIV, infection duration in said subject.

2. The system of claim 1, wherein the porous matrix further comprises, downstream from the first test location, a second test location comprising an immobilized second binding reagent that specifically binds to an anti-viral antibody, e.g., anti-HIV antibody, in said sample liquid having a second average antibody avidity,
wherein relative to said anti-viral antibody, e.g., anti-HIV antibody, in said sample liquid, said second binding reagent is in excess and said anti-viral antibody, e.g., anti-HIV antibody, is limiting, said first average antibody avidity is higher than said second average antibody avidity, and the sample liquid flows laterally along the lateral flow test device and passes the first test location to form a first detectable signal, and to passes the second test location to form a second detectable signal; and each of the first detectable signal and the second detectable signal comprise multiple signal pixels.

3-7. (canceled)

8. The system of claim 1, wherein the first binding reagent specifically binds to an anti-HIV-1 antibody.

9-11. (canceled)

12. The system of claim 1, wherein the first binding reagent comprises a polypeptide that specifically binds to an anti-HIV-1 antibody.

13-15. (canceled)

16. The system of claim 1, wherein the first binding reagent specifically binds to an anti-HIV-2 antibody.

17-19. (canceled)

20. The system of claim 16, wherein the first binding reagent comprises a polypeptide that specifically binds to an anti-HIV-2 antibody.

21-26. (canceled)

27. The system of claim 1, wherein the second binding reagent specifically binds to an anti-HIV-1 antibody.

28-30. (canceled)

31. The system of claim 1, wherein the second binding reagent comprises a polypeptide that specifically binds to an anti-HIV-1 antibody.

32-34. (canceled)

35. The system of claim 1, wherein the second binding reagent specifically binds to an anti-HIV-2 antibody.

36-38. (canceled)

39. The system of claim 35, wherein the second binding reagent comprises a polypeptide that specifically binds to an anti-HIV-2 antibody.

40-42. (canceled)

43. The system of claim 1, wherein both the first binding reagent and the second binding reagent specifically bind to an antibody against the same type of HIV.

44-49. (canceled)

50. The system of claim 1, wherein the amount of the immobilized first binding reagent at the first test location is different from the amount of the second binding reagent at the second test location.

51-54. (canceled)

55. The system of claim 1, wherein the ratio between the distance from the bottom of the sample application pad to the first test location and the distance from the bottom of the sample application pad to the second test location is from about 0.8 to about 0.9.

56. The system of claim 1, wherein the first binding reagent specifically binds to an anti-HIV antibody having a first average antibody avidity from about 0.25 normalized OD units (ODn) to about 6.0 ODn as measured by a HIV-1 Limiting Antigen Avidity EIA described in Duong et at., PLoS ONE, 7(3):e33328 (2012).

57-62. (canceled)

63. The system of claim 1, wherein a portion of the matrix, upstream from the first test location, comprises a dried, labeled reagent, the labeled reagent being capable of being moved by a liquid sample and/or a further liquid to the first test location, the second test location, and/or the control location to generate a detectable signal.

64-65. (canceled)

66. The system of claim 1, wherein the lateral flow test device further comprises, upstream from the first test location, a conjugate element that comprises a dried, labeled reagent, the labeled reagent being capable of moved by a liquid sample and/or a further liquid to the first test location, the second test location, and/or the control location to generate a detectable signal.

67-68. (canceled)

69. The system of claim 63, wherein the labeled reagent binds, and preferably specifically binds, to an anti-viral antibody, e.g., an anti-HIV antibody, in the sample.

70-77. (canceled)

78. The system of claim 1, wherein the reader comprises an image sensor.

79. The system of claim 78, wherein the image sensor is an active pixel sensor.

80-85. (canceled)

86. The system of claim 1, wherein the first quantitative signal readout uses integrated pixel density unit (IPDU).

87. (canceled)

88. The system of claim 1, wherein the second quantitative signal readout uses integrated pixel density unit (IPDU).

89-94. (canceled)

95. The system of claim 1, which is configured for assessing HIV infection duration from about 10 days to about 450 days.

96. A method for assessing viral, e.g., HIV, infection duration in a subject, which method comprises:
a) contacting a sample from a subject with a system of any of claims 1-95, wherein said liquid sample is applied to a site of said lateral flow test device upstream of said first test location;
b) transporting an anti-viral antibody, e.g., an anti-HIV antibody, if present in said liquid sample, and a labeled reagent to said first test location to form a first detectable signal at said first test location, said first detectable signal comprising multiple signal pixels; and
c) measuring both the number of said signal pixels and the intensities of said signal pixels in said first detectable signal using said reader to generate a first quantitative signal; and
d) assessing average antibody avidity of said anti-viral antibody, e.g., anti-HIV antibody, in said sample liquid and/or viral, e.g., HIV, infection duration in said subject based on said first quantitative signal.

97-128. (canceled)
